(19) United States
(12) Patent Application Publication
(10) Pub. No.: US 2026/0275293 A1
(43) Pub. Date: Sep. 17, 2026

(54) Use of Chymopapain in Cell Detachment

(71) Applicants: NOVOZYMES A/S, Bagsvaerd (DK); NOVO NORDISK PHARMATECH A/S, Koege (DK)

(72) Inventors: Tine HOFF, Bagsvaerd (DK); Lars Kobberoee SKOV, Bagsvaerd (DK); Karina Sandgaard KRISTENSEN, Koege (DK); Anna Verena REISER, Bagsvaerd (DK); Casper WILKENS, Koege (DK)

(73) Assignees: NOVOZYMES A/S, Bagsvaerd (DK); NOVO NORDISK PHARMATECH A/S, Koege (DK)

(21) Appl. No.: 19/080,115

(22) Filed: Mar. 14, 2025

(30) Foreign Application Priority Data
Mar. 12, 2024 (EP) 24163084.7

(51) Int. Cl. C12N 5/077; C12N 9/50

(52) U.S. Cl. CPC C12N 5/0657; C12N 9/63; C12Y 304/22006; C12N 2509/00

(57) ABSTRACT

The present invention relates to use of a polypeptide having a sequence identity of at least 70% to SEQ ID NO:1, a mature polypeptide of SEQ ID NO:1, SEQ ID NO:2, or SEQ ID NO:3, wherein the polypeptide has chymopapain activity, in a cell detachment process. The present invention also relates to a method of cell detachment, comprising contacting a cell with a polypeptide having a sequence identity of at least 70% to SEQ ID NO:1, a mature polypeptide of SEQ ID NO:1, SEQ ID NO:2, or SEQ ID NO:3, wherein the polypeptide has chymopapain activity, and wherein the cell is attached to a surface and/or to another cell. The present invention further relates to compositions suitable for cell detachment comprising said polypeptide, polynucleotides encoding said polypeptide, recombinant host cells transformed with said polypeptide, and methods of producing said polypeptide.

### CROSS-REFERENCE TO RELATED APPLICATIONS

This application claims priority or the benefit under 35 U.S.C. 119 of European application no. 24163084.7 filed Mar. 12, 2024. The disclosure of the priority application is fully incorporated herein by reference.

### REFERENCE TO SEQUENCE LISTING

This application contains a Sequence Listing in computer readable form. The contents of the electronic sequence listing created on Mar. 14, 2025, named SQ.xml and 7.87 KB in size, is hereby incorporated by reference in its entirety.

### FIELD OF THE INVENTION

The present invention relates to use of a polypeptide having a sequence identity of at least 70% to SEQ ID NO:1, a mature polypeptide of SEQ ID NO:1, SEQ ID NO:2, or SEQ ID NO:3, wherein the polypeptide has chymopapain activity, in a cell detachment process. The present invention also relates to a method of cell detachment, comprising contacting a cell with a polypeptide having a sequence identity of at least 70% to SEQ ID NO:1, a mature polypeptide of SEQ ID NO:1, SEQ ID NO:2, or SEQ ID NO:3, wherein the polypeptide has chymopapain activity, and wherein the cell is attached to a surface and/or to another cell. The present invention further relates to compositions suitable for cell detachment comprising said polypeptide, polynucleotides encoding said polypeptide, recombinant host cells transformed with said polypeptide, and methods of producing said polypeptide.

### BACKGROUND OF THE INVENTION

Cell detachment is a critical step during passaging of cells when grown as adherent cells as well as cell clusters. The detachment step preferably involves the use of proteolytic enzymes as these are mild yet effective in terms of releasing the cells from a surface to which they adhere as well as dissolving cell clusters formed in suspension cultures.

Accutase® and Accumax® (both available from, e.g., Innovative Cell Technologies, Inc.) are commercially available products for cell detachment that include a mixture of enzymes with proteolytic and collagenolytic activity that are isolated from an invertebrate source. A disadvantage associated with these products is that regulatory authorities generally do not allow animal-derived products to be used in drug development and production processes, which hampers their applicability for cell therapies. Another disadvantage of these products is the inherent risk of batch-to-batch variation in terms of composition and activity as a consequence of these mixtures being animal-derived, leading to a less well-defined product.

TrypLE™ (available from, e.g., ThermoFisher Scientific) is a commercially available trypsin product that may be used for cell detachment. TrypLE™ is produced recombinantly and is thus not of animal origin. However, a disadvantage associated with TrypLE™ is that not all types of cells are sufficiently detached when subjected to trypsin treatment alone, which limits the broad applicability of this product.

Fischer et al. (Stem Cell Research 2018, vol. 32, pp. 65-72) describes the use of papain obtained from Carica papaya as a useful alternative to Accutase and TrypLE for detachment of cardiomyocytes derived from human induced pluripotent stem cells (hiPSCs).

### SUMMARY OF THE INVENTION

The present inventors have identified chymopapain as a particularly useful protease for detachment of cardiomyocytes. As can be seen from the Examples disclosed herein, chymopapain provides improved detachment of cardiomyocytes when compared to papain as well as Accutase®/Accumax®. Notably, chymopapain detachment is associated with improved cell viability and decreased aggregate formation.

In a first aspect, the present invention relates to use of a polypeptide having a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to SEQ ID NO:1, a mature polypeptide of SEQ ID NO:1, SEQ UD NO:2, or SEQ ID NO:3, wherein the polypeptide has chymopapain activity, in a cell detachment process.

In a second aspect, the present invention relates to a method for cell detachment, comprising contacting a cell with a polypeptide having a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to SEQ ID NO:1, a mature polypeptide of SEQ ID NO:1, SEQ ID NO:2, or SEQ ID NO:3, wherein the polypeptide has chymopapain activity, and wherein the cell is attached to a surface and/or to another cell.

In a third aspect, the present invention relates to a composition suitable for cell detachment comprising a polypeptide having a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to SEQ ID NO:1, a mature polypeptide of SEQ ID NO:1, SEQ ID NO:2, or SEQ ID NO:3, wherein the polypeptide has chymopapain activity.

In a fourth aspect, the present invention relates to a polynucleotide encoding a polypeptide having a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to SEQ ID NO:1, a mature polypeptide of SEQ ID NO:1, SEQ ID NO:2, or SEQ ID NO:3, wherein the polypeptide has chymopapain activity.

In a fifth aspect, the present invention relates to a recombinant host cell transformed with a polynucleotide of the fourth aspect.

In a sixth aspect, the present invention relates to a method of producing a polypeptide, comprising (a) cultivating a host cell of the fifth aspect under conditions suitable for expression of the polypeptide; and (b) recovering the polypeptide.

### Sequence Overview

SEQ ID NO:1 is the chymopapain zymogen.

SEQ ID NO:2 is a mature polypeptide of chymopapain.

SEQ ID NO:3 is a mature polypeptide of chymopapain.

SEQ ID NO:4 is a codon-optimized DNA sequence encoding chymopapain.

SEQ ID NO:5 is an Aspergillus signal peptide.

SEQ ID NO:6 is the Suc-Ala-Ala-Pro-Phe-pNA substrate.

### Definitions

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Cell detachment: The term “cell detachment” (e.g., cardiomyocyte detachment) refers to the process of detaching or releasing smaller groups of cells or even single cells from cell cultures, in particular 2D and 3D cell cultures. 2D cell cultures include adherent cell cultures, wherein the cells are grown as monolayers attached to the surface of a cell culture vessel (e.g., a culture flask or petri dish), and wherein the cells are attached to each other and/or to the surface of the cell culture vessel. 3D cell cultures include suspension cultures, wherein the cells are grown as cell clusters suspended in an agitated growth medium, and wherein cells are attached to each other. 3D cell cultures also include concentrated medium cultures (e.g., agarose cultures or Matrigel cultures) as well as scaffold cultures, wherein cells are grown on a structural scaffold. The terms “cell detachment” and “cell dissociation” are used interchangeably herein.

Chymopapain: Chymopapain is a cysteine protease originally isolated from Carica papaya latex. The chymopapain zymogen (SEQ ID NO:1, UniProt accession number P14080) consists of 352 amino acid residues, with Cys159, His293, and Ans313 forming the catalytic triad. For purposes of the present invention, chymopapain activity (EC 3.4.22.6) may be determined as hydrolytic activity on the Bz-Phe-Val-Arg-pNA substrate according to the Chymopapain Activity Assay described below. Chymopapain activity may be differentiated from papain activity (EC 3.4.22.2) in that papain activity involves hydrolytic activity against all of model substrates Z-Phe-Arg-pNA, Bz-Phe-Val-Arg-pNA, Ac-Phe-Gly-pNA, and Boc-Ala-Ala-Gly-pNA (see Example 2 below).

Coding sequence: The term “coding sequence” means a polynucleotide, which directly specifies the amino acid sequence of a polypeptide. The boundaries of the coding sequence are generally determined by an open reading frame, which begins with a start codon, such as ATG, GTG, or TTG, and ends with a stop codon, such as TAA, TAG, or TGA. The coding sequence may be a genomic DNA, cDNA, synthetic DNA, or a combination thereof.

Control sequences: The term “control sequences” means nucleic acid sequences involved in regulation of expression of a polynucleotide in a specific organism or in vitro. Each control sequence may be native (i.e., from the same gene) or heterologous (i.e., from a different gene) to the polynucleotide encoding the polypeptide, and native or heterologous to each other. Such control sequences include, but are not limited to leader, polyadenylation, prepropeptide, propeptide, signal peptide, promoter, terminator, enhancer, and transcription or translation initiator and terminator sequences. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” means any step involved in the production of a polypeptide including, but not limited to, transcription, post-transcriptional modification, translation, post-translational modification, and secretion.

Expression vector: An “expression vector” refers to a linear or circular DNA construct comprising a DNA sequence encoding a polypeptide, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

Heterologous: The term “heterologous” means, with respect to a host cell, that a polypeptide or nucleic acid does not naturally occur in the host cell. The term “heterologous” means, with respect to a polypeptide or nucleic acid, that a control sequence, e.g., promoter, of a polypeptide or nucleic acid is not naturally associated with the polypeptide or nucleic acid, i.e., the control sequence is from a gene other than the gene encoding the mature polypeptide.

Host Strain or Host Cell: A “host strain” or “host cell” is an organism into which an expression vector, phage, virus, or other DNA construct, including a polynucleotide encoding a polypeptide of the present invention has been introduced. Exemplary host strains are microorganism cells (e.g., bacteria, filamentous fungi, and yeast) capable of expressing the polypeptide of interest and/or fermenting saccharides. The term “host cell” includes protoplasts created from cells.

Isolated: The term “isolated” means a polypeptide, nucleic acid, cell, or other specified material or component that has been separated from at least one other material or component, including but not limited to, other proteins, nucleic acids, cells, etc. An isolated polypeptide, nucleic acid, cell or other material is thus in a form that does not occur in nature. An isolated polypeptide includes, but is not limited to, a culture broth containing the secreted polypeptide expressed in a host cell.

Mature polypeptide: The term “mature polypeptide” means a polypeptide in its mature form following N-terminal processing and/or C-terminal processing (e.g., removal of signal peptide).

Native: The term “native” means a nucleic acid or polypeptide naturally occurring in a host cell.

Nucleic acid: The term “nucleic acid” encompasses DNA, RNA, heteroduplexes, and synthetic molecules capable of encoding a polypeptide. Nucleic acids may be single stranded or double stranded and may be chemically modified. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, and which comprises one or more control sequences operably linked to the nucleic acid sequence.

Operably linked: The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequence.

Passage: The term “passage” refers to the process of removing some or all cells from a culture and transferring the cells into fresh growth medium. Passaging of cells may also be referred to as subculturing. In some embodiments, passaging leads to a single cell suspension.

Purified: The term “purified” means a nucleic acid, polypeptide (e.g., a microbial protease) or cell that is substantially free from other components as determined by analytical techniques well known in the art (e.g., a purified polypeptide or nucleic acid may form a discrete band in an electrophoretic gel, chromatographic eluate, and/or a media subjected to density gradient centrifugation). A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8%, about 99.9%, or more, pure (e.g., percent by weight or on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition.

In one aspect, the term “purified” as used herein refers to the polypeptide (e.g., microbial protease) or cell being essentially free from components (especially insoluble components) from the production organism. In other aspects, the term “purified” refers to the polypeptide being essentially free of insoluble components (especially insoluble components) from the native organism from which it is obtained. In one aspect, the polypeptide is separated from some of the soluble components of the organism and culture medium from which it is recovered. The polypeptide may be purified (i.e., separated) by one or more of the unit operations filtration, precipitation, or chromatography.

Accordingly, the polypeptide (e.g., microbial protease) may be purified such that only minor amounts of other proteins, in particular, other polypeptides, are present. The term “purified” as used herein may refer to removal of other components, particularly other proteins and most particularly other enzymes present in the cell of origin of the polypeptide. The polypeptide may be “substantially pure”, i.e., free from other components from the organism in which it is produced, e.g., a host organism for recombinantly produced polypeptide. In one aspect, the polypeptide is at least 40% pure by weight of the total polypeptide material present in the preparation. In one aspect, the polypeptide is at least 50%, 60%, 70%, 80% or 90% pure by weight of the total polypeptide material present in the preparation (e.g., composition suitable for cell detachment). As used herein, a “substantially pure polypeptide” may denote a polypeptide preparation that contains at most 10%, preferably at most 9%, preferably at most 8%, preferably at most 7%, more preferably at most 6%, more preferably at most 5%, more preferably at most 4%, more preferably at most 3%, more preferably at most 2%, more preferably at most 1%, more preferably at most 0.5, more preferably at most 0.1%, more preferably at most 0.05%, more preferably at most 0.01%, even more preferably at most 0.005%, and most preferably at most 0.001% by weight of other polypeptide material with which the polypeptide is natively or recombinantly associated.

It is, therefore, preferred that the substantially pure polypeptide (e.g., microbial protease) is at least 90% pure, preferably at least 91%, more preferably at least 92% pure, more preferably at least 93% pure, more preferably at least 94% pure, more preferably at least 95% pure, more preferably at least 96% pure, more preferably at least 97% pure, more preferably at least 98% pure, more preferably at least 99% pure, more preferably at least 99.5% pure, more preferably at least 99.9% pure, more preferably at least 99.95%, more preferably at least 99.99% pure, even more preferably at least 99.995% pure, and most preferably at least 99.999% pure by weight of the total polypeptide material present in the preparation (e.g., composition suitable for cell detachment). The polypeptide of the present invention is preferably in a substantially pure form (i.e., the preparation is essentially free of other polypeptide material with which it is natively or recombinantly associated). This can be accomplished, for example by preparing the polypeptide by well-known recombinant methods or by classical purification methods.

Recombinant: The term “recombinant” is used in its conventional meaning to refer to the manipulation, e.g., cutting and rejoining, of nucleic acid sequences to form constellations different from those found in nature. The term recombinant refers to a cell, nucleic acid, polypeptide or vector that has been modified from its native state. Thus, for example, recombinant cells express genes that are not found within the native (non-recombinant) form of the cell, or express native genes at different levels or under different conditions than found in nature. The term “recombinant” is synonymous with “genetically modified” and “transgenic”.

Recover: The terms “recover” or “recovery” means the removal of a polypeptide from at least one fermentation broth component selected from the list of a cell, a nucleic acid, or other specified material, e.g., recovery of the polypeptide from the whole fermentation broth, or from the cell-free fermentation broth, by polypeptide crystal harvest, by chromatography, by filtration, e.g., depth filtration (by use of filter aids or packed filter medias, cloth filtration in chamber filters, rotary-drum filtration, drum filtration, rotary vacuum-drum filters, candle filters, horizontal leaf filters or similar, using sheet or pad filtration in framed or modular setups) or membrane filtration (using sheet filtration, module filtration, candle filtration, microfiltration, ultrafiltration in either cross flow, dynamic cross flow or dead end operation), or by centrifugation (using decanter centrifuges, disc stack centrifuges, hydro cyclones or similar), or by precipitating the polypeptide and using relevant solid-liquid separation methods to harvest the polypeptide from the broth media by use of classification separation by particle sizes. Recovery encompasses isolation and/or purification of the polypeptide.

Sequence identity: The relatedness between two amino acid sequences or between two nucleotide sequences is described by the parameter “sequence identity”.

For purposes of the present invention, the sequence identity between two amino acid sequences is determined as the output of “longest identity” using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the -nobrief option must be specified in the command line. The output of Needle labeled “longest identity” is calculated as follows:

(Identical Residues×100)/(Length of Alignment−Total Number of Gaps in Alignment)

For purposes of the present invention, the sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 6.6.0 or later. The parameters used are a gap open penalty of 10, a gap extension penalty of 0.5, and the EDNAFULL (EMBOSS version of NCBI NUC4.4) substitution matrix. In order for the Needle program to report the longest identity, the nobrief option must be specified in the command line. The output of Needle labeled “longest identity” is calculated as follows:

$$( Identical Deoxyribonucleotides \times 100 ) / ( Length of Alignment - Total Number of Gaps in Alignment )$$

### DETAILED DESCRIPTION OF THE INVENTION

The present inventors have identified chymopapain as a particularly useful protease for detachment of cardiomyocytes. As can be seen from the Examples disclosed herein, chymopapain provides improved detachment of cardiomyocytes when compared to papain as well as Accutase®/Accumax®. Notably, chymopapain detachment is associated with improved cell viability and decreased aggregate formation. Without being bound by theory, it is speculated that the substrate specificity exhibited by chymopapain (see Example 2 below) is particularly compatible with cardiomyocytes and provides an effective yet mild cleavage of cardiomyocyte surface proteins involved in surface attachment and cell-cell adhesion.

### Polypeptides

In one aspect, the present invention relates to a polypeptide having a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to SEQ ID NO:1, a mature polypeptide of SEQ ID NO:1, SEQ ID NO:2, or SEQ ID NO:3, wherein the polypeptide has chymopapain activity.

In one embodiment, the polypeptide has a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to a mature polypeptide of SEQ ID NO:1, wherein the polypeptide has chymopapain activity.

In one embodiment, the polypeptide comprises, consists essentially of, or consists of a mature polypeptide of SEQ ID NO:1.

SEQ ID NO:2 (corresponding to amino acid residues 134 to 352 of SEQ ID NO:1) and SEQ ID NO:3 (corresponding to amino acid residues 135 to 352 of SEQ ID NO:1) are experimentally observed mature polypeptides of SEQ ID NO:1.

In one embodiment, the polypeptide has a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to SEQ ID NO:2, wherein the polypeptide has chymopapain activity.

In one embodiment, the polypeptide comprises, consists essentially of, or consists of SEQ ID NO:2.

In a preferred embodiment, the polypeptide is a fragment or variant of SEQ ID NO:2.

In one embodiment, the polypeptide has a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to SEQ ID NO:3, wherein the polypeptide has chymopapain activity.

In one embodiment, the polypeptide comprises, consists essentially of, or consists of SEQ ID NO:3.

In a preferred embodiment, the polypeptide is a fragment or variant of SEQ ID NO:3.

In a most preferred embodiment, the polypeptide is chymopapain (EC 3.4.22.2).

In another aspect, the polypeptide is derived from SEQ ID NO:1 or a mature polypeptide of SEQ ID NO:1 by substitution, deletion, or addition of one or several amino acids. In some embodiments, the polypeptide is a variant of SEQ ID NO:2 comprising a substitution, deletion, and/or insertion at one or more positions. In some embodiments, the polypeptide is a variant of SEQ ID NO:3 comprising a substitution, deletion, and/or insertion at one or more positions. In one aspect, the number of amino acid substitutions, deletions and/or insertions introduced into SEQ ID NO:1 or a mature polypeptide of SEQ ID NO:1 is up to 15, e.g., 1, 2, 3, 4, 5, 6, 7, 8, 9, 10, 11, 12, 13, 14, or 15. In one aspect, the number of amino acid substitutions, deletions and/or insertions introduced into SEQ ID NO:2 is up to 15, e.g., 1, 2, 3, 4, 5, 6, 7, 8, 9, 10, 11, 12, 13, 14, or 15. In one aspect, the number of amino acid substitutions, deletions and/or insertions introduced into SEQ ID NO:3 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

Essential amino acids in a polypeptide, e.g., a microbial protease, can be identified according to procedures known in the art, such as site-directed mutagenesis or alanine-scanning mutagenesis (Cunningham and Wells, 1989, Science 244:1081-1085). In the latter technique, single alanine mutations are introduced at every residue in the molecule, and the resultant molecules are tested for protease activity and/or P1 specificity to identify amino acid residues that are critical to the activity and/or the specificity of the molecule (see also Hilton et al., 1996, J. Biol. Chem. 271:4699-4708). The active site of a microbial protease can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255:306-312; Smith et al., 1992, J. Mol. Biol. 224:899-904; Wlodaver et al., 1992, FEBS Lett. 309:59-64. The identity of essential amino acids can also be inferred from an alignment with a related polypeptide, and/or be inferred from sequence homology and conserved catalytic machinery with a related polypeptide or within a polypeptide or protein family with polypeptides/proteins descending from a common ancestor, typically having similar three-dimensional structures, functions, and significant sequence similarity. Additionally, or alternatively, protein structure prediction tools can be used for protein structure modelling to identify essential amino acids and/or active sites of polypeptides. See, for example, Jumper et al., 2021, “Highly accurate protein structure prediction with AlphaFold”, Nature 596:583-589.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241:53-57; Bowie and Sauer, 1989, Proc. Natl. Acad. Sci. USA 86:2152-2156; WO 95/17413; or WO 95/22625. Other methods that can be used include error-prone PCR, phage display (e.g., Lowman et al., 1991, Biochemistry 30:10832-10837; U.S. Pat. No. 5,223,409; WO 92/06204), and region-directed mutagenesis (Derbyshire et al., 1986, Gene 46:145; Ner et al., 1988, DNA 7:127).

Mutagenesis/shuffling methods can be combined with high-throughput, automated screening methods to detect activity of cloned, mutagenized polypeptides expressed by host cells (Ness et al., 1999, Nature Biotechnology 17:893-896). Mutagenized DNA molecules that encode active polypeptides can be recovered from the host cells and rapidly sequenced using standard methods in the art. These methods allow the rapid determination of the importance of individual amino acid residues in a polypeptide.

In one aspect, the polypeptide is isolated.

In another aspect, the polypeptide is purified.

### Polynucleotides

In one aspect, the present invention relates to polynucleotides encoding a polypeptide of the present invention.

The polynucleotide may be a genomic DNA, a cDNA, a synthetic DNA, a synthetic RNA, a mRNA, or a combination thereof. The polynucleotide may be cloned Carica papaya.

The polynucleotide may also be mutated by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2:95-107.

In an aspect, the polynucleotide is isolated.

In another aspect, the polynucleotide is purified.

### Nucleic Acid Constructs

The present invention also relates to nucleic acid constructs comprising a polynucleotide of the present invention, wherein the polynucleotide is operably linked to one or more control sequences that direct the expression of the coding sequence in a suitable host cell under conditions compatible with the control sequences.

The polynucleotide may be manipulated in a variety of ways to provide for expression of a polypeptide of the invention. Manipulation of the polynucleotide prior to its insertion into a vector may be desirable or necessary depending on the expression vector. Techniques for modifying polynucleotides utilizing recombinant DNA methods are well known in the art.

### Promoters

The control sequence may be a promoter, a polynucleotide that is recognized by a host cell for expression of a polynucleotide encoding a polypeptide of the invention. The promoter contains transcriptional control sequences that mediate the expression of polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the polynucleotide of the present invention in a bacterial host cell are described in Sambrook et al., 1989, Molecular Cloning: A Laboratory Manual, Cold Spring Harbor Lab., NY, Davis et al., 2012, supra, and Song et al., 2016, PLoS One 11(7):e0158447.

Examples of suitable promoters for directing transcription of the polynucleotide of the present invention in a filamentous fungal host cell are promoters obtained from Aspergillus, Fusarium, Rhizomucor and Trichoderma cells, such as the promoters described in Mukherjee et al., 2013, “Trichoderma: Biology and Applications”, and by Schmoll and Dattenbock, 2016, “Gene Expression Systems in Fungi: Advancements and Applications”, Fungal Biology.

For expression in a yeast host, examples of useful promoters are described by Smolke et al., 2018, “Synthetic Biology: Parts, Devices and Applications” (Chapter 6: Constitutive and Regulated Promoters in Yeast: How to Design and Make Use of Promoters in S. cerevisiae), and by Schmoll and Dattenböck, 2016, “Gene Expression Systems in Fungi: Advancements and Applications”, Fungal Biology.

### Terminators

The control sequence may also be a transcription terminator, which is recognized by a host cell to terminate transcription. The terminator is operably linked to the 3′-terminus of the polynucleotide encoding a polypeptide of the invention. Any terminator that is functional in the host cell may be used in the present invention.

Preferred terminators for bacterial host cells may be obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells may be obtained from Aspergillus or Trichoderma species, such as obtained from the genes for Aspergillus niger glucoamylase, Trichoderma reesei beta-glucosidase, Trichoderma reesei cellobiohydrolase I, and Trichoderma reesei endoglucanase I, such as the terminators described in Mukherjee et al., 2013, “Trichoderma: Biology and Applications”, and by Schmoll and Dattenböck, 2016, “Gene Expression Systems in Fungi: Advancements and Applications”, Fungal Biology.

Preferred terminators for yeast host cells may be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, Yeast 8:423-488.

mRNA Stabilizers

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, J. Bacteriol. 177:3465-3471).

Examples of mRNA stabilizer regions for fungal cells are described in Geisberg et al., 2014, Cell 156 (4): 812-824, and in Morozov et al., 2006, Eukaryotic Cell 5(11):1838-1846.

### Leader Sequences

The control sequence may also be a leader, a non-translated region of an mRNA that is important for translation by the host cell. The leader is operably linked to the 5′-terminus of the polynucleotide encoding a polypeptide of the invention. Any leader that is functional in the host cell may be used.

Suitable leaders for bacterial host cells are described by Hambraeus et al., 2000, Microbiology 146(12):3051-3059, and by Kaberdin and Bläsi, 2006, FEMS Microbiol. Rev. 30(6): 967-979.

Preferred leaders for filamentous fungal host cells may be obtained from the genes for Aspergillus oryzae TAKA amylase and Aspergillus nidulans triose phosphate isomerase.

Suitable leaders for yeast host cells may be obtained from the genes for Saccharomyces cerevisiae enolase (ENO-1), Saccharomyces cerevisiae 3-phosphoglycerate kinase, alcohol Saccharomyces cerevisiae alpha-factor, and Saccharomyces cerevisiae dehydrogenase/glyceraldehyde-3-phosphate dehydrogenase (ADH2/GAP).

### Polyadenylation Sequences

The control sequence may also be a polyadenylation sequence, a sequence operably linked to the 3′-terminus of the polynucleotide which, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15:5983-5990

### Signal Peptides

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes a polypeptide of the invention. Alternatively, the 5′-end of the coding sequence may contain a signal peptide coding sequence that is heterologous to the coding sequence. A heterologous signal peptide coding sequence may be required where the coding sequence does not naturally contain a signal peptide coding sequence. Alternatively, a heterologous signal peptide coding sequence may simply replace the natural signal peptide coding sequence to enhance secretion of a polypeptide of the invention. Any signal peptide coding sequence that directs the expressed microbial protease into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Freudl, 2018, Microbial Cell Factories 17:52.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase, such as the signal peptide described by Xu et al., 2018, Biotechnology Letters 40:949-955.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

### Propeptides

The control sequence may also be a propeptide coding sequence that encodes a propeptide positioned at the N-terminus of a polypeptide of the invention. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence. Additionally, or alternatively, when both signal peptide and propeptide sequences are present, the polypeptide may comprise only a part of the signal peptide sequence and/or only a part of the propeptide sequence. Alternatively, the final or isolated polypeptide may comprise a mixture of mature polypeptides and polypeptides which comprise, either partly or in full length, a propeptide sequence and/or a signal peptide sequence.

### Regulatory Sequences

It may also be desirable to add regulatory sequences that regulate expression of a polypeptide of the invention relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In fungal systems, these regulatory sequences include the dihydrofolate reductase gene that is amplified in the presence of methotrexate, and the metallothionein genes that are amplified with heavy metals.

### Transcription Factors

The control sequence may also be a transcription factor, a polynucleotide encoding a polynucleotide-specific DNA-binding polypeptide that controls the rate of the transcription of genetic information from DNA to mRNA by binding to a specific polynucleotide sequence. The transcription factor may function alone and/or together with one or more other polypeptides or transcription factors in a complex by promoting or blocking the recruitment of RNA polymerase. Transcription factors are characterized by comprising at least one DNA-binding domain which often attaches to a specific DNA sequence adjacent to the genetic elements which are regulated by the transcription factor. The transcription factor may regulate the expression of a protein of interest either directly, i.e., by activating the transcription of the gene encoding the protein of interest by binding to its promoter, or indirectly, i.e., by activating the transcription of a further transcription factor which regulates the transcription of the gene encoding the protein of interest, such as by binding to the promoter of the further transcription factor. Suitable transcription factors for fungal host cells are described in WO 2017/144177. Suitable transcription factors for prokaryotic host cells are described in Seshasayee et al., 2011, Subcellular Biochemistry 52:7-23, as well in Balleza et al., 2009, FEMS Microbiol. Rev. 33(1): 133-151.

### Expression Vectors

In one aspect, the present invention relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding a polypeptide of the present invention at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

The vector preferably contains at least one element that permits integration of the vector into the host cell's genome or autonomous replication of the vector in the cell independent of the genome.

For integration into the host cell genome, the vector may rely on the polynucleotide's sequence encoding the microbial protease or any other element of the vector for integration into the genome by homologous recombination, such as homology-directed repair (HDR), or non-homologous recombination, such as non-homologous end-joining (NHEJ).

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

More than one copy of a polynucleotide of the present invention may be inserted into a host cell to increase production of a polypeptide of the invention. For example, 2 or 3 or 4 or 5 or more copies are inserted into a host cell. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

### Recombinant Host Cells

In one aspect, the present invention relates to recombinant host cells comprising a polynucleotide of the present invention operably linked to one or more control sequences that direct the production of a polypeptide of the present invention.

In one embodiment, the polypeptide has a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to SEQ ID NO:1, a mature polypeptide of SEQ ID NO:1, SEQ ID NO:2, or SEQ ID NO:3, wherein the polypeptide has chymopapain activity.

In one embodiment, the polypeptide has a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to a mature polypeptide of SEQ ID NO:1, wherein the polypeptide has chymopapain activity.

In one embodiment, the polypeptide comprises, consists essentially of, or consists of a mature polypeptide of SEQ ID NO:1.

In one embodiment, the polypeptide has a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to SEQ ID NO:2, wherein the polypeptide has chymopapain activity.

In one embodiment, the polypeptide comprises, consists essentially of, or consists of SEQ ID NO:2.

In a preferred embodiment, the polypeptide is a fragment or variant of SEQ ID NO:2.

In one embodiment, the polypeptide has a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to SEQ ID NO:3, wherein the polypeptide has chymopapain activity.

In one embodiment, the polypeptide comprises, consists essentially of, or consists of SEQ ID NO:3.

In a preferred embodiment, the polypeptide is a fragment or variant of SEQ ID NO:3.

In a most preferred embodiment, the polypeptide is chymopapain (EC 3.4.22.2).

A construct or vector comprising a polynucleotide is introduced into a host cell so that the construct or vector is maintained as a chromosomal integrant or as a self-replicating extrachromosomal vector as described earlier. The choice of a host cell will to a large extent depend upon the gene encoding the polypeptide of the invention and its source. The polypeptide of the invention may be native or heterologous to the recombinant host cell. Also, at least one of the one or more control sequences can be heterologous to the polynucleotide encoding the polypeptide of the invention. The recombinant host cell may comprise a single copy, or at least two copies, e.g., three, four, five, or more copies of the polynucleotide of the present invention.

The host cell may be any microbial cell useful in the recombinant production of a polypeptide of the invention, e.g., a prokaryotic cell or a fungal cell.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells. In an embodiment, the Bacillus cell is a Bacillus amyloliquefaciens, Bacillus licheniformis, or Bacillus subtilis cell.

For purposes of this invention, Bacillus classes/genera/species shall be defined as described in Patel and Gupta, 2020, Int. J. Syst. Evol. Microbiol. 70:406-438.

The bacterial host cell may also be any Streptococcus cell including, but not limited to, Streptococcus equisimilis, Streptococcus pyogenes, Streptococcus uberis, and Streptococcus equi subsp. zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

Methods for introducing DNA into prokaryotic host cells are well-known in the art, and any suitable method can be used including but not limited to protoplast transformation, competent cell transformation, electroporation, conjugation, transduction, with DNA introduced as linearized or as circular polynucleotide. Persons skilled in the art will be readily able to identify a suitable method for introducing DNA into a given prokaryotic cell depending, e.g., on the genus. Methods for introducing DNA into prokaryotic host cells are for example described in Heinze et al., 2018, BMC Microbiology 18:56, Burke et al., 2001, Proc. Natl. Acad. Sci. USA 98:6289-6294, Choi et al., 2006, J. Microbiol. Methods 64:391-397, and Donald et al., 2013, J. Bacteriol. 195(11):2612-2620.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

Fungal cells may be transformed by a process involving protoplast-mediated transformation, Agrobacterium-mediated transformation, electroporation, biolistic method and shock-wave-mediated transformation as reviewed by Li et al., 2017, Microbial Cell Factories 16:168 and procedures described in EP 238023, Yelton et al., 1984, Proc. Natl. Acad. Sci. USA 81:1470-1474, Christensen et al., 1988, Bio/Technology 6:1419-1422, and Lubertozzi and Keasling, 2009, Biotechn. Advances 27:53-75. However, any method known in the art for introducing DNA into a fungal host cell can be used, and the DNA can be introduced as linearized or as circular polynucleotide.

The fungal host cell may be a yeast cell. “Yeast” as used herein includes ascosporogenous yeast (Endomycetales), basidiosporogenous yeast, and yeast belonging to the Fungi Imperfecti (Blastomycetes). For purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell. In a preferred embodiment, the yeast host cell is a Pichia or Komagataella cell, e.g., a Pichia pastoris cell (Komagataella phaffii).

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell. In a preferred embodiment, the filamentous fungal host cell is an Aspergillus, Trichoderma or Fusarium cell. In a further preferred embodiment, the filamentous fungal host cell is an Aspergillus niger, Aspergillus oryzae, Trichoderma reesei, or Fusarium venenatum cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

In a most preferred embodiment, the filamentous fungal host cell is an Aspergillus cell, preferably an Aspergillus niger cell or an Aspergillus host cell.

In an aspect, the host cell is isolated.

In another aspect, the host cell is purified.

### Methods of Production

In one aspect, the present invention relates to methods of producing a polypeptide of the present invention of the present invention, comprising (a) cultivating a host cell, which in its wild-type form produces a polypeptide of the invention under conditions conducive for production of said polypeptide; and optionally, (b) recovering the polypeptide.

In one embodiment, the polypeptide has a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to SEQ ID NO:1, a mature polypeptide of SEQ ID NO:1, SEQ ID NO:2, or SEQ ID NO:3, wherein the polypeptide has chymopapain activity.

In one embodiment, the polypeptide has a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to a mature polypeptide of SEQ ID NO:1, wherein the polypeptide has chymopapain activity.

In one embodiment, the polypeptide comprises, consists essentially of, or consists of a mature polypeptide of SEQ ID NO:1.

In one embodiment, the polypeptide has a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to SEQ ID NO:2, wherein the polypeptide has chymopapain activity.

In one embodiment, the polypeptide comprises, consists essentially of, or consists of SEQ ID NO:2.

In a preferred embodiment, the polypeptide is a fragment or variant of SEQ ID NO:2.

In one embodiment, the polypeptide has a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to SEQ ID NO:3, wherein the polypeptide has chymopapain activity.

In one embodiment, the polypeptide comprises, consists essentially of, or consists of SEQ ID NO:3.

In a preferred embodiment, the polypeptide is a fragment or variant of SEQ ID NO:3.

In a most preferred embodiment, the polypeptide is chymopapain (EC 3.4.22.2).

In one aspect, the present invention relates to methods of producing a polypeptide of the invention, comprising (a) cultivating a recombinant host cell of the present invention under conditions conducive for production of the polypeptide; and optionally, (b) recovering the polypeptide.

In one embodiment, the polypeptide has a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to SEQ ID NO:1, a mature polypeptide of SEQ ID NO:1, SEQ ID NO:2, or SEQ ID NO:3, wherein the polypeptide has chymopapain activity.

In one embodiment, the polypeptide has a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to a mature polypeptide of SEQ ID NO:1, wherein the polypeptide has chymopapain activity.

In one embodiment, the polypeptide comprises, consists essentially of, or consists of a mature polypeptide of SEQ ID NO:1.

In one embodiment, the polypeptide has a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to SEQ ID NO:2, wherein the polypeptide has chymopapain activity.

In one embodiment, the polypeptide comprises, consists essentially of, or consists of SEQ ID NO:2.

In a preferred embodiment, the polypeptide is a fragment or variant of SEQ ID NO:2.

In one embodiment, the polypeptide has a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to SEQ ID NO:3, wherein the polypeptide has chymopapain activity.

In one embodiment, the polypeptide comprises, consists essentially of, or consists of SEQ ID NO:3.

In a preferred embodiment, the polypeptide is a fragment or variant of SEQ ID NO:3.

In a most preferred embodiment, the polypeptide is chymopapain (EC 3.4.22.2).

The recombinant host cell may be a bacterial or fungal host cell. In a preferred embodiment, the recombinant host cell is a Bacillus cell, most preferably a B. subtilis cell or a B. licheniformis cell. In a preferred embodiment, the recombinant host cell is an Aspergillus cell, most preferably an A. niger cell or an A. oryzae cell. In a preferred embodiment, the recombinant host cell is a Pichia cell, most preferably a P. pastoris cell. In a most preferred embodiment, the recombinant host cell is an A. oryzae cell.

The host cell is cultivated in a nutrient medium suitable for production of a polypeptide of the invention using methods known in the art. For example, the cell may be cultivated by shake flask cultivation, or small-scale or large-scale fermentation (including continuous, batch, fed-batch, or solid-state, and/or microcarrier-based fermentations) in laboratory or industrial fermentors in a suitable medium and under conditions allowing of the polypeptide of the invention to be expressed and/or isolated. Suitable media are available from commercial suppliers or may be prepared according to published compositions (e.g., in catalogues of the American Type Culture Collection). If the polypeptide of the invention is secreted into the nutrient medium, the polypeptide may be recovered directly from the medium. If the polypeptide is not secreted, it can be recovered from cell lysates.

The polypeptides of the present invention may be detected using methods known in the art that are specific for the polypeptide, including, but not limited to, the use of specific antibodies, formation of an enzyme product, disappearance of an enzyme substrate, or an assay determining the relative or specific activity of the polypeptide.

The polypeptides of the present invention may be recovered from the medium using methods known in the art, including, but not limited to, collection, centrifugation, filtration, extraction, spray-drying, freeze-drying, evaporation, or precipitation. In one aspect, a whole fermentation broth comprising the polypeptide of the invention is recovered. In another aspect, a cell-free fermentation broth comprising the polypeptide of the invention is recovered.

The polypeptide of the invention may be purified by a variety of procedures known in the art to obtain a substantially pure polypeptides and/or polypeptide fragments (see, e.g., Wingfield, 2015, Current Protocols in Protein Science; 80(1): 6.1.1-6.1.35; Labrou, 2014, Protein Downstream Processing, 1129:3-10).

In an alternative aspect, the polypeptide of the present invention is not recovered.

### Protease Granules

In one aspect, the present invention relates to enzyme granules/particles comprising a polypeptide of the present invention. In an embodiment, the granule comprises a core, and optionally one or more coatings (outer layers) surrounding the core.

The core may have a diameter, measured as equivalent spherical diameter (volume based average particle size), of 20-2000 μm, particularly 50-1500 μm, 100-1500 μm or 250-1200 μm. The core diameter, measured as equivalent spherical diameter, can be determined using laser diffraction, such as using a Malvern Mastersizer and/or the method described under ISO13320 (2020).

In one embodiment, the polypeptide has a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to SEQ ID NO:1, a mature polypeptide of SEQ ID NO:1, SEQ ID NO:2, or SEQ ID NO:3, wherein the polypeptide has chymopapain activity.

In one embodiment, the polypeptide has a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to a mature polypeptide of SEQ ID NO:1, wherein the polypeptide has chymopapain activity.

In one embodiment, the polypeptide comprises, consists essentially of, or consists of a mature polypeptide of SEQ ID NO:1.

In one embodiment, the polypeptide has a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to SEQ ID NO:2, wherein the polypeptide has chymopapain activity.

In one embodiment, the polypeptide comprises, consists essentially of, or consists of SEQ ID NO:2.

In a preferred embodiment, the polypeptide is a fragment or variant of SEQ ID NO:2.

In one embodiment, the polypeptide has a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to SEQ ID NO:3, wherein the polypeptide has chymopapain activity.

In one embodiment, the polypeptide comprises, consists essentially of, or consists of SEQ ID NO:3.

In a preferred embodiment, the polypeptide is a fragment or variant of SEQ ID NO:3.

In a most preferred embodiment, the polypeptide is chymopapain (EC 3.4.22.2).

The core may include additional materials such as fillers, fiber materials (cellulose or synthetic fibers), stabilizing agents, solubilizing agents, suspension agents, viscosity regulating agents, light spheres, plasticizers, salts, lubricants and fragrances.

The core may include a binder, such as synthetic polymer, wax, fat, or carbohydrate.

The core may include a salt of a multivalent cation, a reducing agent, an antioxidant, a peroxide decomposing catalyst and/or an acidic buffer component, typically as a homogenous blend.

The core may include an inert particle with the polypeptide absorbed into it, or applied onto the surface, e.g., by fluid bed coating.

The core may have a diameter of 20-2000 μm, particularly 50-1500 μm, 100-1500 μm or 250-1200 μm.

The core may be surrounded by at least one coating, e.g., to improve the storage stability, to reduce dust formation during handling, or for coloring the granule. The optional coating(s) may include a salt coating, or other suitable coating materials, such as polyethylene glycol (PEG), methyl hydroxy-propyl cellulose (MHPC) and polyvinyl alcohol (PVA).

The coating may be applied in an amount of at least 0.1% by weight of the core, e.g., at least 0.5%, at least 1%, at least 5%, at least 10%, or at least 15%. The amount may be at most 100%, 70%, 50%, 40% or 30%.

The coating is preferably at least 0.1 μm thick, particularly at least 0.5 μm, at least 1 μm or at least 5 μm. In some embodiments, the thickness of the coating is below 100 μm, such as below 60 μm, or below 40 μm.

The coating should encapsulate the core unit by forming a substantially continuous layer. A substantially continuous layer is to be understood as a coating having few or no holes, so that the core unit has few or no uncoated areas. The layer or coating should, in particular, be homogeneous in thickness.

The coating can further contain other materials as known in the art, e.g., fillers, antisticking agents, pigments, dyes, plasticizers and/or binders, such as titanium dioxide, kaolin, calcium carbonate or talc.

A salt coating may comprise at least 60% by weight of a salt, e.g., at least 65%, at least 70%, at least 75%, at least 80%, at least 85%, at least 90%, at least 95% or at least 99% by weight.

To provide acceptable protection, the salt coating is preferably at least 0.1 μm thick, e.g., at least 0.5 μm, at least 1 μm, at least 2 μm, at least 4 μm, at least 5 μm, or at least 8 μm. In a particular embodiment, the thickness of the salt coating is below 100 μm, such as below 60 μm, or below 40 μm.

The salt may be added from a salt solution where the salt is completely dissolved or from a salt suspension wherein the fine particles are less than 50 μm, such as less than 10 μm or less than 5 μm.

The salt coating may comprise a single salt or a mixture of two or more salts. The salt may be water soluble, in particular, having a solubility at least 0.1 g in 100 g of water at 20° C., preferably at least 0.5 g per 100 g water, e.g., at least 1 g per 100 g water, e.g., at least 5 g per 100 g water.

The salt may be an inorganic salt, e.g., salts of sulfate, sulfite, phosphate, phosphonate, nitrate, chloride or carbonate or salts of simple organic acids (less than 10 carbon atoms, e.g., 6 or less carbon atoms) such as citrate, malonate or acetate. Examples of cations in these salts are alkali or earth alkali metal ions, the ammonium ion or metal ions of the first transition series, such as sodium, potassium, magnesium, calcium, zinc or aluminum. Examples of anions include chloride, bromide, iodide, sulfate, sulfite, bisulfite, thiosulfate, phosphate, monobasic phosphate, dibasic phosphate, hypophosphite, dihydrogen pyrophosphate, tetraborate, borate, carbonate, bicarbonate, metasilicate, citrate, malate, maleate, malonate, succinate, lactate, formate, acetate, butyrate, propionate, benzoate, tartrate, ascorbate or gluconate. In particular, alkali- or earth alkali metal salts of sulfate, sulfite, phosphate, phosphonate, nitrate, chloride or carbonate or salts of simple organic acids such as citrate, malonate or acetate may be used.

The salt in the coating may have a constant humidity at 20° C. above 60%, particularly above 70%, above 80% or above 85%, or it may be another hydrate form of such a salt (e.g., anhydrate). The salt coating may be as described in WO 00/01793 or WO 2006/034710.

Specific examples of suitable salts are NaCl (CH20° C.=76%), Na₂CO₃ (CH20° C.=92%), NaNO₃ (CH20° C.=73%), Na₂HPO₄ (CH20° C.=95%), Na₃PO₄ (CH25° C.=92%), NH₄Cl (CH20° C.=79.5%), (NH₄)₂HPO₄ (CH20° C.=93.0%), NH₄H₂PO₄ (CH20° C.=93.1%), (NH₄)₂SO₄ (CH20° C.=81.1%), KCl (CH20° C.=85%), K₂HPO₄ (CH20° C.=92%), KH₂PO₄ (CH20° C.=96.5%), KNO₃ (CH20° C.-93.5%), Na₂SO₄ (CH20° C.=93%), K₂SO₄ (CH20° C.=98%), KHSO₄ (CH20° C.=86%), MgSO₄ (CH20° C.=90%), ZnSO₄ (CH20° C.=90%) and sodium citrate (CH25° C.=86%). Other examples include NaH₂PO₄, (NH₄)H₂PO₄, CuSO₄, Mg(NO₃)₂ and magnesium acetate.

The salt may be in anhydrous form, or it may be a hydrated salt, i.e., a crystalline salt hydrate with bound water(s) of crystallization, such as described in WO 99/32595. Specific examples include anhydrous sodium sulfate (Na₂SO₄), anhydrous magnesium sulfate (MgSO₄), magnesium sulfate heptahydrate (MgSO₄·7H₂O), zinc sulfate heptahydrate (ZnSO₄·7H₂O), sodium phosphate dibasic heptahydrate (Na₂HPO₄·7H₂O), magnesium nitrate hexahydrate (Mg(NO₃)₂(6H₂O)), sodium citrate dihydrate and magnesium acetate tetrahydrate.

Preferably the salt is applied as a solution of the salt, e.g., using a fluid bed.

The coating materials can be waxy coating materials and film-forming coating materials. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591.

The granule may optionally have one or more additional coatings. Examples of suitable coating materials are polyethylene glycol (PEG), methyl hydroxy-propyl cellulose (MHPC) and polyvinyl alcohol (PVA). Examples of enzyme granules with multiple coatings are described in WO 93/07263 and WO 97/23606.

The core can be prepared by granulating a blend of the ingredients, e.g., by a method comprising granulation techniques such as crystallization, precipitation, pan-coating, fluid bed coating, fluid bed agglomeration, rotary atomization, extrusion, prilling, spheronization, size reduction methods, drum granulation, and/or high shear granulation.

Methods for preparing the core can be found in the Handbook of Powder Technology; Particle size enlargement by C. E. Capes; Vol. 1; 1980; Elsevier. Preparation methods include known feed and granule formulation technologies, e.g.,
(a) Spray dried products, wherein a liquid polypeptide-containing solution is atomized in a spray drying tower to form small droplets which during their way down the drying tower dry to form a microbial protease-containing particulate material. Very small particles can be produced this way (Michael S. Showell (editor); Powdered detergents; Surfactant Science Series; 1998; Vol. 71; pages 140-142; Marcel Dekker).
(b) Layered products, wherein the polypeptide of the invention is coated as a layer around a pre-formed inert core particle, wherein a polypeptide-containing solution is atomized, typically in a fluid bed apparatus wherein the pre-formed core particles are fluidized, and the polypeptide-containing solution adheres to the core particles and dries up to leave a layer of dry polypeptide on the surface of the core particle. Particles of a desired size can be obtained this way if a useful core particle of the desired size can be found. This type of product is described in, e.g., WO 97/23606.
(c) Absorbed core particles, wherein rather than coating the polypeptide of the invention as a layer around the core, the polypeptide of the invention is absorbed onto and/or into the surface of the core. Such a process is described in WO 97/39116.
(d) Extrusion or pelletized products, wherein a polypeptide-containing paste is pressed to pellets or under pressure is extruded through a small opening and cut into particles which are subsequently dried. Such particles usually have a considerable size because of the material in which the extrusion opening is made (usually a plate with bore holes) sets a limit on the allowable pressure drop over the extrusion opening. Also, very high extrusion pressures when using a small opening increase heat generation in the polypeptide paste, which is harmful to the polypeptide (Michael S. Showell (editor); Powdered detergents; Surfactant Science Series; 1998; Vol. 71; pages 140-142; Marcel Dekker).
(e) Prilled products, wherein a polypeptide-containing powder is suspended in molten wax and the suspension is sprayed, e.g., through a rotating disk atomizer, into a cooling chamber where the droplets quickly solidify (Michael S. Showell (editor); Powdered detergents; Surfactant Science Series; 1998; Vol. 71; pages 140-142; Marcel Dekker). The product obtained is one wherein the polypeptide is uniformly distributed throughout an inert material instead of being concentrated on its surface. U.S. Pat. Nos. 4,016,040 and 4,713,245 describe this technique.
(f) Mixer granulation products, wherein a polypeptide-containing liquid is added to a dry powder composition of conventional granulating components. The liquid and the powder in a suitable proportion are mixed and as the moisture of the liquid is absorbed in the dry powder, the components of the dry powder will start to adhere and agglomerate and particles will build up, forming granulates comprising the polypeptide. Such a process is described in U.S. Pat. No. 4,106,991, EP 170360, EP 304332, EP 304331, WO 90/09440 and WO 90/09428. In a particular aspect of this process, various high-shear mixers can be used as granulators. Granulates consisting of polypeptide of the invention, fillers and binders etc. are mixed with cellulose fibers to reinforce the particles to produce a so-called T-granulate. Reinforced particles are more robust and release less enzymatic dust.
(g) Size reduction, wherein the cores are produced by milling or crushing of larger particles, pellets, tablets, briquettes, etc. containing the polypeptide of the invention. The wanted core particle fraction is obtained by sieving the milled or crushed product. Over and undersized particles can be recycled. Size reduction is described in Martin Rhodes (editor); Principles of Powder Technology; 1990; Chapter 10; John Wiley & Sons.
(h) Fluid bed granulation. Fluid bed granulation involves suspending particulates in an air stream and spraying a liquid onto the fluidized particles via nozzles. Particles hit by spray droplets get wetted and become tacky. The tacky particles collide with other particles and adhere to them to form a granule.
(i) The cores may be subjected to drying, such as in a fluid bed drier. Other known methods for drying granules in the feed or enzyme industry can be used by the skilled person. The drying preferably takes place at a product temperature of from 25 to 90° C. For some polypeptides, it is important that the cores comprising the polypeptide contain a low amount of water before coating with the salt. If a water-sensitive polypeptide is coated with a salt before excessive water is removed, the excessive water will be trapped within the core and may affect the activity of the polypeptide negatively. After drying, the cores preferably contain 0.1-10% w/w water.

Non-dusting granulates may be produced, e.g., as disclosed in U.S. Pat. Nos. 4,106,991 and 4,661,452 and may optionally be coated by methods known in the art.

The granulate may further comprise one or more additional enzymes, e.g., hydrolase, isomerase, ligase, lyase, oxidoreductase, and transferase. The one or more additional enzymes are preferably selected from the group consisting of acetylxylan esterase, acylglycerol lipase, amylase, alpha-amylase, beta-amylase, arabinofuranosidase, cellobiohydrolases, cellulase, feruloyl esterase, galactanase, alpha-galactosidase, beta-galactosidase, beta-glucanase, beta-glucosidase, lysophospholipase, lysozyme, alpha-mannosidase, beta-mannosidase (mannanase), phytase, phospholipase A1, phospholipase A2, phospholipase D, pullulanase, pectin esterase, triacylglycerol lipase, xylanase, beta-xylosidase or any combination thereof. Each enzyme will then be present in more granules securing a more uniform distribution of the enzymes, and also reduces the physical segregation of different enzymes due to different particle sizes. Methods for producing multi-enzyme co-granulates is disclosed in the ip.com disclosure IPCOM000200739D.

Another example of formulation of polypeptides by the use of co-granulates is disclosed in WO 2013/188331.

The present invention also relates to protected polypeptides prepared according to the method disclosed in EP 238216.

### Fermentation Broth Formulations or Cell Compositions

In one aspect, the present invention relates to a fermentation broth formulation or a cell composition comprising a polypeptide of the present invention. The fermentation broth formulation or the cell composition further comprises additional ingredients used in the fermentation process, such as, for example, cells (including, the host cells containing the gene encoding a polypeptide of the invention which are used to produce said polypeptide), cell debris, biomass, fermentation media and/or fermentation products. In some embodiments, the composition is a cell-killed whole broth containing organic acid(s), killed cells and/or cell debris, and culture medium.

In one embodiment, the polypeptide has a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to SEQ ID NO:1, a mature polypeptide of SEQ ID NO:1, SEQ ID NO:2, or SEQ ID NO:3, wherein the polypeptide has chymopapain activity.

In one embodiment, the polypeptide has a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to a mature polypeptide of SEQ ID NO:1, wherein the polypeptide has chymopapain activity.

In one embodiment, the polypeptide comprises, consists essentially of, or consists of a mature polypeptide of SEQ ID NO:1.

In one embodiment, the polypeptide has a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to SEQ ID NO:2, wherein the polypeptide has chymopapain activity.

In one embodiment, the polypeptide comprises, consists essentially of, or consists of SEQ ID NO:2.

In a preferred embodiment, the polypeptide is a fragment or variant of SEQ ID NO:2.

In one embodiment, the polypeptide has a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to SEQ ID NO:3, wherein the polypeptide has chymopapain activity.

In one embodiment, the polypeptide comprises, consists essentially of, or consists of SEQ ID NO:3.

In a preferred embodiment, the polypeptide is a fragment or variant of SEQ ID NO:3.

In a most preferred embodiment, the polypeptide is chymopapain (EC 3.4.22.2).

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In some embodiments, the fermentation broth formulation or the cell composition comprises a first organic acid component comprising at least one 1-5 carbon organic acid and/or a salt thereof and a second organic acid component comprising at least one 6 or more carbon organic acid and/or a salt thereof. In some embodiments, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In some embodiments, the killed cells and/or cell debris are removed from a cell-killed whole broth to provide a composition that is free of these components.

The fermentation broth formulation or cell composition may further comprise a preservative and/or anti-microbial (e.g., bacteriostatic) agent, including, but not limited to, sorbitol, sodium chloride, potassium sorbate, and others known in the art.

The cell-killed whole broth or cell composition may contain the unfractionated contents of the fermentation materials derived at the end of the fermentation. Typically, the cell-killed whole broth or cell composition contains the spent culture medium and cell debris present after the microbial cells (e.g., filamentous fungal cells) are grown to saturation, incubated under carbon-limiting conditions to allow protein synthesis. In some embodiments, the cell-killed whole broth or cell composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673

### Compositions Suitable for Cell Detachment

In one aspect, the present invention relates to compositions suitable for cell detachment comprising a polypeptide having a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to SEQ ID NO:1, a mature polypeptide of SEQ ID NO:1, SEQ ID NO:2, or SEQ ID NO:3, wherein the polypeptide has chymopapain activity.

In one embodiment, the polypeptide has a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to a mature polypeptide of SEQ ID NO:1, wherein the polypeptide has chymopapain activity.

In one embodiment, the polypeptide comprises, consists essentially of, or consists of a mature polypeptide of SEQ ID NO:1.

In one embodiment, the polypeptide has a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to SEQ ID NO:2, wherein the polypeptide has chymopapain activity.

In one embodiment, the polypeptide comprises, consists essentially of, or consists of SEQ ID NO:2.

In a preferred embodiment, the polypeptide is a fragment or variant of SEQ ID NO:2.

In one embodiment, the polypeptide has a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to SEQ ID NO:3, wherein the polypeptide has chymopapain activity.

In one embodiment, the polypeptide comprises, consists essentially of, or consists of SEQ ID NO:3.

In a preferred embodiment, the polypeptide is a fragment or variant of SEQ ID NO:3.

In a most preferred embodiment, the polypeptide is chymopapain (EC 3.4.22.2).

In one embodiment, the polypeptide has a purity of at least 90%, e.g., at least 91%, at least 92%, at least 93%, at least 94%, at least 95%, at least 96%, at least 97%, at least 98%, at least 99%, at least 99.5%, at least 99.9%, at least 99.95%, at least 99.99%, at least 99.995%, at least 99.999%, or more, by weight of the total polypeptide material present in the composition.

In a preferred embodiment, the polypeptide has a purity of at least 99%, e.g., at least 99.5%, least 99.9%, at least 99.95%, at least 99.99%, at least 99.995%, at least 99.999%, or more, by weight of the total polypeptide material present in the composition.

In a most preferred embodiment, the polypeptide has a purity of at least 99.9%, e.g., at least 99.95%, at least 99.99%, at least 99.995%, at least 99.999%, or more, by weight of the total polypeptide material present in the composition.

In some embodiments, the composition suitable for cell detachment is a liquid composition. Preferably, the composition is an aqueous composition to ensure compatibility with media commonly used for cell cultures. In some embodiments, the composition is a freeze-dried liquid composition. In an alternative embodiment, the composition is a solid composition.

To ensure that the liquid composition has a pH value that is compatible with cell culturing conditions, the composition may comprise an aqueous buffer. The composition may comprise aqueous buffer in an amount of 1-99% by weight, e.g., 5-95%, 10-90%, 15-85%, 20-80%, or 25-75% aqueous buffer. Alternatively, the composition may comprise at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, at least 99.5%, or more, aqueous buffer by weight.

In some embodiments, the liquid composition has a pH value of about 5 to about 9, e.g., pH 5, pH 5.5, pH 6, pH 6.5, pH 7, pH 7.5, pH 8, pH 8.5, or pH 9. More preferably, the composition has a pH value of about 7 to about 8, e.g., pH 7, pH 7.1, pH 7.2, pH 7.3, pH 7.4, pH 7.5, pH 7.6, pH 7.7, pH, 7.8, pH 7.9, or pH 8. Even more preferably, the composition has a pH value of about 7 to about 7.5, e.g., pH 7.1, pH 7.2, pH 7.3, pH 7.4, or pH 7.5. Most preferably, the composition has a pH value of about 7.4.

In some embodiments, the aqueous buffer comprises 4-(2-hydroxyethyl)-1-piperazineethanesulfonic acid (HEPES), tris(hydroxymethyl)aminomethane (TRIS), phosphate, or bicarbonate. Preferably, the aqueous buffer is a HEPES buffer, a TRIS buffer, or a phosphate (e.g., PBS) buffer.

In some embodiments, the liquid composition comprises a microbial protease of the invention in an amount of from about 0.1 μg/ml to about 100 g/ml, e.g., from about 0.5 μg/ml to about 50 μg/ml, from about 1 μg/ml to about 20 g/ml, or from about 1 μg/ml to about 10 μg/ml.

In some embodiments, the liquid composition comprises a microbial protease of the invention in an amount of from about 0.1 μg/ml to about 20 μg/ml, e.g., about 0.1 μg/ml, about 0.2 μg/ml, about 0.3 μg/ml, about 0.4 μg/ml, about 0.5 μg/ml, about 0.6 μg/ml, about 0.7 μg/ml, about 0.8 μg/ml, about 0.9 μg/ml, about 1 μg/ml, about 2 μg/ml, about 3 μg/ml, about 4 μg/ml about 5 μg/ml, about 6 μg/ml, about 7 μg/ml, about 8 μg/ml, about 9 μg/ml, about 10 μg/ml, about 11 μg/ml, about 12 μg/ml, about 13 μg/ml, about 14 μg/ml, about 15 μg/ml, about 16 μg/ml, about 17 μg/ml, about 18 μg/ml, about 19 μg/ml, or about 20 μg/ml.

In some embodiments, the liquid composition comprises a microbial protease of the invention in an amount of from about 0.5 μg/ml to about 5 μg/ml, e.g., about 0.5 μg/ml, about 0.6 μg/ml, about 0.7 μg/ml, about 0.8 μg/ml, about 0.9 μg/ml, about 1 μg/ml, about 2 μg/ml, about 3 μg/ml, or about 4 μg/ml, or about 5 μg/ml.

In some embodiments, the liquid composition comprises a microbial protease of the invention in an amount of from about 1 μg/ml to about 10 μg/ml, e.g., about 1 μg/ml, about 2 μg/ml, about 3 μg/ml, about 4 μg/ml, about 5 μg/ml, about 6 μg/ml, about 7 μg/ml, about 8 μg/ml, about 9 μg/ml, or about 10 μg/ml.

In some embodiments, the liquid composition comprises a microbial protease of the invention in an amount of from about 1 μg/ml to about 20 μg/ml, e.g., about 1 μg/ml, about 2 μg/ml, about 3 μg/ml, about 4 μg/ml, about 5 μg/ml, about 6 μg/ml, about 7 μg/ml, about 8 μg/ml, about 9 μg/ml, about 10 μg/ml, about 11 μg/ml, about 12 μg/ml, about 13 μg/ml, about 14 μg/ml, about 15 μg/ml, about 16 μg/ml, about 17 μg/ml, about 18 μg/ml, about 19 μg/ml, or about 20 μg/ml.

In a preferred embodiment, the liquid composition comprises a microbial protease of the invention in an amount of from 1 μg/ml to 20 μg/ml, e.g., 1 μg/ml, 2 μg/ml, 3 μg/ml, 4 g/ml, 5 μg/m, 6 μg/ml, 7 μg/ml, 8 μg/ml, 9 μg/ml, 10 μg/ml, 11 μg/ml, 12 μg/ml, 13 μg/ml, 14 μg/ml, 15 μg/ml, 16 μg/ml, 17 μg/ml, 18 μg/ml, 19 μg/ml, or 20 μg/ml, more preferably from 1 μg/ml to 10 μg/ml, most preferably from 1 μg/ml to 5 μg/ml.

In some embodiments, the liquid composition comprises ethylenediaminetetraacetic acid (EDTA). Preferably, the liquid composition comprises EDTA in an amount of from about 0.01 mM to about 100 mM, e.g., from about 0.05 mM to about 50 mM, from about 0.1 mM to about 10 mM, or from about 0.5 mM to about 5 mM. Preferably, the liquid composition comprises EDTA in an amount of about 0.1 mM, about 0.2 mM, about 0.3 mM, about 0.3 mM, about 0.4 mM, about 0.5 mM, about 0.6 mM, about 0.7 mM, about 0.8 mM, about 0.9 mM, about 0.95 mM, about 1 mM, about 1.5 mM, about 2 mM, about 3 mM, about 4 mM, about 5 mM, about 6 mM, about 7 mM, about 8 mM, about 9 mM, or about 10 mM. More preferably, liquid composition comprises EDTA in an amount of about 0.5 mM, about 0.6 mM, about 0.7 mM, about 0.8 mM, about 0.9 mM, about 0.95 mM, about 1 mM, about 1.5 mM, about 2 mM, about 3 mM, about 4 mM, or about 5 mM. Most preferably, the liquid composition comprises EDTA in an amount of about 1 mM.

In some embodiments, the liquid composition comprises substantially no magnesium ions (Mg²⁺) and/or calcium ions (Ca²⁺). In some embodiments, the liquid composition does not comprise magnesium ions (Mg²⁺) and/or calcium ions (Ca²⁺). In some embodiments, the liquid does not comprise magnesium ions (Mg²⁺) or calcium ions (Ca²⁺).

In some embodiments, the liquid composition comprises a phosphate buffer (e.g., PBS), EDTA, and substantially no magnesium ions (Mg²⁺) and/or calcium ions (Ca²⁺).

In a preferred embodiment, the liquid composition comprises a phosphate buffer (e.g., PBS) having a pH value of about 7 to about 8, preferably of about 7 to about 7.5, most preferably of about pH 7.4; wherein the liquid composition further comprises EDTA in an amount of from about 0.1 mM to about 10 mM, preferably from about 0.5 mM to about 5 mM, most preferably of about 1 mM; and wherein the liquid composition comprises substantially no magnesium ions (Mg²⁺) and/or calcium ions (Ca²⁺).

In a preferred embodiment, the liquid composition comprises a phosphate buffer (e.g., PBS) having a pH value of about 7 to about 7.5, most preferably of about pH 7.4; wherein the liquid composition further comprises EDTA in an amount of from about 0.5 mM to about 5 mM, most preferably of about 1 mM; and wherein the liquid composition does not comprise magnesium ions (Mg²⁺) or calcium ions (Ca²⁺).

In a preferred embodiment, the liquid composition comprises a phosphate buffer (e.g., PBS) having a pH value of about 7 to about 7.5, most preferably of about pH 7.4; wherein the liquid composition further comprises EDTA in an amount of from about 0.5 mM to about 5 mM, most preferably of about 1 mM; wherein the liquid composition does not comprise magnesium ions (Mg²⁺) or calcium ions (Ca²⁺); and wherein the composition comprises SEQ ID NO:1 or SEQ ID NO:2 in an amount of from 0.1 μg/ml to 20 g/ml.

In a preferred embodiment, the liquid composition comprises a phosphate buffer (e.g., PBS) having a pH value of about pH 7.4; wherein the composition further comprises EDTA in an amount of about 1 mM; and wherein the composition does not comprise magnesium ions (Mg²⁺) or calcium ions (Ca²⁺).

In a preferred embodiment, the liquid composition comprises a phosphate buffer (e.g., PBS) having a pH value of about pH 7.4; wherein the composition further comprises EDTA in an amount of about 1 mM; wherein the composition does not comprise magnesium ions (Mg²⁺) or calcium ions (Ca²⁺); and wherein the composition comprises SEQ ID NO:1 or SEQ ID NO:2 in an amount of from 1 μg/ml to 20 μg/ml.

The liquid composition may further comprise an enzyme stabilizer (examples of which include polyols such as propylene glycol or glycerol, sugar or sugar alcohol, lactic acid, reversible protease inhibitor, boric acid, or a boric acid derivative, e.g., an aromatic borate ester, or a phenyl boronic acid derivative such as 4-formylphenyl boronic acid).

In some embodiments, filler(s) or carrier material(s) are included to increase the volume of the liquid composition. Suitable filler or carrier materials include, but are not limited to, various salts of sulfate, carbonate and silicate as well as talc, clay and the like. Suitable filler or carrier materials for liquid compositions include, but are not limited to, water or low molecular weight primary and secondary alcohols including polyols and diols. Examples of such alcohols include, but are not limited to, methanol, ethanol, propanol and isopropanol. In some embodiments, the compositions contain from about 5% to about 90% of such materials.

In an aspect, the liquid composition comprises 20-80% w/w of polyol. In one embodiment, the liquid composition comprises 0.001-2% w/w preservative.

In another embodiment, the invention relates to liquid compositions comprising:
(a) 0.001-25% w/w of a polypeptide having a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to SEQ ID NO:1 and having chymopapain activity (EC 3.4.22.6); preferably wherein the polypeptide comprises, consists essentially of, or consists of SEQ ID NO:1;
(b) 20-80% w/w of polyol;
(c) optionally 0.001-2% w/w preservative; and
(d) water.

In another embodiment, the invention relates to liquid compositions comprising:
(a) 0.001-25% w/w of a polypeptide having a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to SEQ ID NO:1 and having chymopapain activity (EC 3.4.22.6); preferably wherein the polypeptide comprises, consists essentially of, or consists of SEQ ID NO:1;
(b) 0.001-2% w/w preservative;
(c) optionally 20-80% w/w of polyol; and
(d) water.

In another embodiment, the liquid composition comprises one or more formulating agents, such as a formulating agent selected from the group consisting of polyol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch, PVA, acetate and phosphate, preferably selected from the group consisting of sodium sulfate, dextrin, cellulose, sodium thiosulfate, kaolin and calcium carbonate. In one embodiment, the polyols is selected from the group consisting of glycerol, sorbitol, propylene glycol (MPG), ethylene glycol, diethylene glycol, triethylene glycol, 1,2-propylene glycol or 1,3-propylene glycol, dipropylene glycol, polyethylene glycol (PEG) having an average molecular weight below about 600 and polypropylene glycol (PPG) having an average molecular weight below about 600, more preferably selected from the group consisting of glycerol, sorbitol and propylene glycol (MPG) or any combination thereof.

In one embodiment, the liquid composition comprises glucose in an amount of from about 0.1 g/L to about 10 g/L, e.g., about 0.1 g/L, about 0.2 g/L, about 0.3 g/L, about 0.4 g/L, about 0.5 g/L, about 0.6 g/L, about 0.7 g/L, about 0.8 g/L, about 0.9 g/L, about 1 g/L, about 2 g/L, about 3 g/L, about 4 g/L, about 5 g/L, about 6 g/L, about 7 g/L, about 8 g/L, about 9 g/L, or about 10 g/L. In a preferred embodiment, the liquid composition comprises glucose in an amount of from about 0.5 g/L to about 5 g/L, most preferably in an amount of about 1 g/L.

In another embodiment, the liquid composition comprises 20-80% polyol (i.e., total amount of polyol), e.g., 25-75% polyol, 30-70% polyol, 35-65% polyol, or 40-60% polyol. In one embodiment, the liquid formulation comprises 20-80% polyol, e.g., 25-75% polyol, 30-70% polyol, 35-65% polyol, or 40-60% polyol, wherein the polyol is selected from the group consisting of glycerol, sorbitol, propylene glycol (MPG), ethylene glycol, diethylene glycol, triethylene glycol, 1,2-propylene glycol or 1,3-propylene glycol, dipropylene glycol, polyethylene glycol (PEG) having an average molecular weight below about 600 and polypropylene glycol (PPG) having an average molecular weight below about 600. In one embodiment, the liquid formulation comprises 20-80% polyol (i.e., total amount of polyol), e.g., 25-75% polyol, 30-70% polyol, 35-65% polyol, or 40-60% polyol, wherein the polyol is selected from the group consisting of glycerol, sorbitol and propylene glycol (MPG).

In another embodiment, the preservative is selected from the group consisting of sodium sorbate, potassium sorbate, sodium benzoate and potassium benzoate or any combination thereof. In one embodiment, the liquid composition comprises 0.02-1.5% w/w preservative, e.g., 0.05-1% w/w preservative or 0.1-0.5% w/w preservative. In one embodiment, the liquid formulation composition 0.001-2% w/w preservative (i.e., total amount of preservative), e.g., 0.02-1.5% w/w preservative, 0.05-1% w/w preservative, or 0.1-0.5% w/w preservative, wherein the preservative is selected from the group consisting of sodium sorbate, potassium sorbate, sodium benzoate and potassium benzoate or any combination thereof.

In one aspect, the composition further comprises one or more additional enzymes, e.g., hydrolase, isomerase, ligase, lyase, oxidoreductase, and transferase. The one or more additional enzymes are preferably selected from the group consisting of acetylxylan esterase, acylglycerol lipase, amylase, alpha-amylase, beta-amylase, arabinofuranosidase, cellobiohydrolases, cellulase, DNase, feruloyl esterase, galactanase, alpha-galactosidase, beta-galactosidase, beta-glucanase, beta-glucosidase, lysophospholipase, lysozyme, alpha-mannosidase, beta-mannosidase (mannanase), phytase, phospholipase A1, phospholipase A2, phospholipase D, pullulanase, pectin esterase, triacylglycerol lipase, xylanase, beta-xylosidase or any combination thereof. In a preferred embodiment, the composition further comprises a DNAse.

### Methods and Uses

In one aspect, the present invention relates to methods for cell detachment, comprising contacting a cell with a polypeptide of the invention, wherein the cell is attached to a surface and/or to another cell.

In one embodiment, the polypeptide has a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to SEQ ID NO:1, a mature polypeptide of SEQ ID NO:1, SEQ ID NO:2, or SEQ ID NO:3, wherein the polypeptide has chymopapain activity.

In one embodiment, the polypeptide has a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to a mature polypeptide of SEQ ID NO:1, wherein the polypeptide has chymopapain activity.

In one embodiment, the polypeptide comprises, consists essentially of, or consists of a mature polypeptide of SEQ ID NO:1.

In one embodiment, the polypeptide has a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to SEQ ID NO:2, wherein the polypeptide has chymopapain activity.

In one embodiment, the polypeptide comprises, consists essentially of, or consists of SEQ ID NO:2.

In a preferred embodiment, the polypeptide is a fragment or variant of SEQ ID NO:2.

In one embodiment, the polypeptide has a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to SEQ ID NO:3, wherein the polypeptide has chymopapain activity.

In one embodiment, the polypeptide comprises, consists essentially of, or consists of SEQ ID NO:3.

In a preferred embodiment, the polypeptide is a fragment or variant of SEQ ID NO:3.

In a most preferred embodiment, the polypeptide is chymopapain (EC 3.4.22.2).

In one embodiment, the cell to be detached is attached to a surface, e.g., a plastic surface or a glass surface. In one embodiment, the cell to be detached is part of a cell monolayer. In one embodiment, the cell to be detached is part of a cell cluster.

In some embodiments, the cell to be detached is attached to a surface coated with biomaterials, extra cellular matrix (ECM), and/or other scaffolds fabricated from natural polymers (e.g., collagen, hyaluronic acid, fibrin, alginate, gelatine, etc.) or synthetic polymers (e.g., poly(glycolic acid) (PGA), poly(lactic acid) (PLA), poly(lactic-co-glycolic acid) (PLGA), and polycaprolactone (PCL), etc.).

In a preferred embodiment, the cell to be detached is a cardiomyocyte. In a more preferred embodiment, the cell is a mammalian cardiomyocyte. In a most preferred embodiment, the cell is a human cardiomyocyte.

In one embodiment, the cardiomyocyte, preferably a human cardiomyocyte, is attached to a surface, e.g., a plastic surface or a glass surface.

In one embodiment, the cardiomyocyte, preferably a human cardiomyocyte, is part of a cell monolayer.

In one embodiment, the cardiomyocyte, preferably a human cardiomyocyte, is part of a cell cluster.

In some embodiments, the cardiomyocyte to be detached is attached to a surface coated with biomaterials, extra cellular matrix (ECM), and/or other scaffolds fabricated from natural polymers (e.g., collagen, hyaluronic acid, fibrin, alginate, gelatine, etc.) or synthetic polymers (e.g., poly(glycolic acid) (PGA), poly(lactic acid) (PLA), poly(lactic-co-glycolic acid) (PLGA), and polycaprolactone (PCL), etc.).

In one aspect, the present invention also relates to use of a polypeptide having a sequence identity of at least 70%, e.g., at least 75%, at least 80%, at least 85%, at least 90%, at least 91%, at least 92%, at least 93%, at least 94%, at least 95%, at least 96%, at least 97%, at least 98%, at least 99%, or even 100%, to SEQ ID NO:1 and having chymopapain activity (EC 3.4.22.6) in a cell detachment process. In one embodiment, the polypeptide comprises, consists essentially of, or consists of SEQ ID NO:1. In a preferred embodiment, the polypeptide is chymopapain or a fragment or variant thereof. In a most preferred embodiment, the polypeptide is chymopapain (SEQ ID NO:1).

In one embodiment, the cell to be detached is attached to a surface, e.g., a plastic surface or a glass surface. In one embodiment, the cell to be detached is part of a cell monolayer. In one embodiment, the cell to be detached is part of a cell cluster.

In some embodiments, the cell to be detached is attached to a surface coated with biomaterials, extra cellular matrix (ECM), and/or other scaffolds fabricated from natural polymers (e.g., collagen, hyaluronic acid, fibrin, alginate, gelatine, etc.) or synthetic polymers (e.g., poly(glycolic acid) (PGA), poly(lactic acid) (PLA), poly(lactic-co-glycolic acid) (PLGA), and polycaprolactone (PCL), etc.).

In a preferred embodiment, the cell to be detached is a cardiomyocyte. In a more preferred embodiment, the cell is a mammalian cardiomyocyte. In a most preferred embodiment, the cell is a human cardiomyocyte.

In one embodiment, the cardiomyocyte, preferably a human cardiomyocyte, is attached to a surface, e.g., a plastic surface or a glass surface.

In one embodiment, the cardiomyocyte, preferably a human cardiomyocyte, is part of a cell monolayer.

In one embodiment, the cardiomyocyte, preferably a human cardiomyocyte, is part of a cell cluster.

In some embodiments, the cardiomyocyte to be detached is attached to a surface coated with biomaterials, extra cellular matrix (ECM), and/or other scaffolds fabricated from natural polymers (e.g., collagen, hyaluronic acid, fibrin, alginate, gelatine, etc.) or synthetic polymers (e.g., poly(glycolic acid) (PGA), poly(lactic acid) (PLA), poly(lactic-co-glycolic acid) (PLGA), and polycaprolactone (PCL), etc.).

### EXAMPLES

### Materials and Methods

### Enzymes

Chymopapain was produced recombinantly according to Example 1. Papain was purchased as Carica papaya papain (Roche 10108014001) or Papaya latex papain (Merck P4762) and used as is. DNase I (Roche 04536282001) was purchased from Sigma-Aldrich and used as is. Accutase® and Accumax® were purchased from Innovative Cell Technologies and used as is.

### Evaluation of Cardiomyocyte Cluster Dissociation in 3D

Human pluripotent stem cell-derived cardiomyocyte (hPSC-CM) clusters were cultured in suspension format. At the CM08 stage (eight days after differentiation), 3 mL cluster suspension was harvested by centrifugation (300 g; 1 min) and the supernatant medium removed. The cells were subsequently washed with 3 mL PBS without Ca²⁺ and Mg²⁺, centrifugated (300 g; 1 min) and the supernatant was removed. The enzyme solution was prepared by dissolving SEQ ID NO:1 and Carica papaya papain (Roche 10108014001) in Dulbecco's PBS buffer without Ca²⁺ and Mg²⁺; to a concentration of 17 and 100 μg/mL, respectively followed by preheating to room temperature and addition of 0.5 mM EDTA. One mL enzyme solution was added to the CM clusters, and the clusters were incubated (70 rpm; 37° C.) until the medium became turbid with single cells. Accumax® (a 3× formulation of Accutase®, Innovative Cell Technologies) was added in a similar way. The clusters were ensured fully disintegrated, and 2 mL culture medium (preheated to 37° C.) was added to stop the enzymatic reaction. DNAse I (Roche 04536282001, 50 U/mL final concentration) was added to the stop media when cells were treated with Carica papaya papain (Roche 10108014001).

After adding culture medium, images were taken and cell count was performed to determine total live and dead cell numbers, cell viability, single cell diameter, debris index, and fraction of (unwanted) aggregates (not fully dissociated into single-cell suspension) using a NucleoCounter NC-202 automated cell counter [1, 2].

### Evaluation of Cardiomyocyte Dissociation in 2D

Human embryonic stem cell-derived cardiomyocytes (hESC-CMs) were expanded in 6-well cell culture plates coated with Matrigel similar to Poon et al. (Cell Res. 2020, vol. 30, pp. 626-629). Late-stage CMs (3 passages) were dissociated by replacing the medium with 0.5 mL preheated (37° C.) 10 μg/mL SEQ ID NO:1 or Papaya latex papain (Merck) in Dulbecco's PBS buffer without Ca2+ and Mg2+ supplemented with 0.5 mM EDTA. The reaction was stopped after 10 min by adding 0.5 mL Roswell Park Memorial Institute (RPMI) medium supplemented with 10% fetal bovine serum (FBS) and the cells were resuspended in RPMI medium supplemented with B27+ and Rock Inhibitor (RI) after harvesting. After detachment, images were taken and the detached cells were analyzed to determined total amounts of cells, cell viability, single cell diameter, and fraction of aggregates using a NucleoCounter NC-200 according to manufacturer's instructions.

### Example 1: Recombinant Expression and Purification of Chymopapain

Recombinant Expression in Aspergillus oryzae

A codon-optimized DNA sequence encoding chymopapain (SEQ ID NO:4) was used to construct an expression vector for Aspergillus. The Aspergillus expression vector consists of an expression cassette based on the Aspergillus niger neutral amylase II promoter fused to the Aspergillus nidulans triose phosphate isomerase non-translated leader sequence (Pna2/tpi) and the Aspergillus niger amyloglucosidase terminator (Tamg). A pyrG selective marker from Aspergillus nidulans was used for transformant selection. An Aspergillus signal peptide (MKLSWLVAAALTAASVVSA, SEQ ID NO:5) was employed instead of the native chymopapain signal peptide.

The expression plasmid for chymopapain was transformed into Aspergillus oryzae as described in Lassen et al. (2001), Applied and Environmental Microbiology, Vol. 67, No. 10. Four transformants were isolated, purified, and cultivated in microtiter plates. Expression was determined using SDS-PAGE analysis, and the best producing strain was further fermented in shake flasks.

### Purification of Recombinant Chymopapain

The fermentation supernatant containing chymopapain was filtered through a Fast PES Bottle top filter with a 0.22 μm cut-off, and the pH of the resulting solution was adjusted to 6.0 with acetic acid. Chymopapain was subsequently purified by chromatography on SP Sepharose, approximately 30 ml in a XK26 column, using as buffer A 50 mM MES, 2 mM CaCl₂, pH 6.0, and as buffer B 50 mM MES, 2 mM CaCl₂, 1 M NaCl, pH 6.0. Fractions were analyzed by SDS-PAGE and pooled based on the chromatogram (monitoring absorption at 260 and 280 nm) and SDS-PAGE analysis with addition of the HALT inhibitor (Thermo Scientific, Cat. No. 78429).

The molecular weight, as estimated by SDS-PAGE, was approximately 23 kDa and the purity was >90%.

### Chymopapain Activity Assay

Chymopapain activity may be determined using Bz-Phe-Val-Arg-pNA (Bachem) as substrate. A stock substrate solution is made by dissolving 30 mg of Bz-Phe-Val-Arg-pNA in 5 ml dimethyl sulfoxide (DSMO). The stock substrate solution is diluted 10 times with 100 mM MES buffer, pH 6.0, and 170 μl of diluted substrate solution is dispensed into each well of a microtiter plate and preheated for 15 minutes at 37° C. The chymopapain solution is diluted to an appropriate level (within the linear range of the concentration curve) in 100 mM MES, 100 mM L-cysteine, pH 6.0, and 30 μl of diluted chymopapain solution is added to the diluted substrate solution in the wells of the plate. After a short mixing (5 seconds), absorption at 405 nm (A₄₀₅) is measured every 30 seconds for 30 minutes at 37° C.

### Example 2: Substrate Specificity of Chymopapain, Papain, and Accutase®

The substrate specificities of chymopapain, Papaya latex papain and Accutase® were evaluated using several commercially available model substrates (see Table 1). Stock substrate solutions were made by dissolving 30 mg model substrate in 5 ml dimethyl sulfoxide (DSMO). The stock substrate solutions were diluted 10 times with 100 mM MES buffer, pH 6.0, and 170 μl of diluted substrate solution was dispensed into each well of a microtiter plate and preheated for 15 minutes at 37° C. The enzyme solutions were diluted to an appropriate level (within the linear range of the concentration curve) in 100 mM MES, 100 mM L-cysteine, pH 6.0, and 30 μl of diluted enzyme solution was added to the diluted substrate solutions in the wells of the plate. After a short mixing (5 seconds), absorption at 405 nm (A₄₀₅) is measured every 30 seconds for 30 minutes at 37° C.

As can be deduced from Table 1, chymopapain exhibits a narrow substrate specificity against the Bz-Phe-Val-Arg-pNA model substrate, whereas papain and Accutase® display activity against a wider range of substrates.

TABLE 1
Substrate specificities for Chymopapain, Papain and Accutase.

| | Suc- | | | | | | | | | |
|---|---|---|---|---|---|---|---|---|---|---|
| | Ala- | Suc- | H- | Tos- | Z- | H- | | Bz- | | Boc- |
| | Ala- | Gly- | Ala- | Gly- | Gly- | Glu- | Z- | Phe- | Ac- | Ala- |
| | Pro- | Gly- | Ala- | Pro- | Pro- | Gly- | Phe- | Val- | Phe- | Ala- |
| | Phe- | Phe- | Phe- | Lys- | Arg- | Arg- | Arg- | Arg- | Gly- | Gly- |
| | pNA* | pNA | pNA | pNA | pNA | pNA | pNA | pNA | pNA | Pna |
| Chymopapain | − | − | − | − | − | − | − | + | − | − |
| Papaya latex papain | − | − | − | − | − | − | + | + | + | + |
| Accutase ® | + | − | − | + | + | + | + | + | − | − |

*SEQ ID NO: 6

### Example 3: Cardiomyocyte Cluster Dissociation and Re-Formation (3D)

Chymopapain, Carica papaya papain, and Accumax® were evaluated for CM8 cluster dissociation (Table 2). Treatment with papain alone resulted in formation of a large gel-like aggregate that could not be resuspended after harvesting. Resuspension of papain-treated cells was only possible if the cells were co-treated with DNAse by addition of DNase I to the stop medium. In contrast, resuspension of cells following chymopapain treatment did not require additional treatment with DNase and also resulted in higher yield (total cells) and reduced aggregate formation compared to papain+DNase treatment. Compared to Accutase, chymopapain treatment resulted in improved cell viability and reduced aggregate formation.

TABLE 2
Evaluation of CM8 clusters dissociation

| | | | | Carica |
|---|---|---|---|---|
| | | | Carica | papaya |
| | | | papaya | papain + |
| | Accumax ® | Chymopapain | papain | DNase |
| Total cells | 1.39 × 10⁶ | 1.22 × 10⁶ | N.D. | 1.05 × 10⁶ |
| (cells/mL) | | | | |
| Live cells | 1.22 × 10⁶ | 1.11 × 10⁶ | N.D. | 9.84 × 10⁵ |
| (cells/mL | | | | |
| Cell viability (%) | 87 | 91 | N.D. | 93 |
| Cell diameter (μm) | 17 | 17 | N.D. | 17 |
| Aggregates (%) | 5 | 3 | 100* | 11 |
| Debris index | 35 | 40 | N.D. | 45 |

N.D. = not detected;
*Based on visual inspection.

### Example 4: Cardiomyocyte Dissociation in 2D

Chymopapain and Papaya latex a pain were evaluated for late-stage CM dissociation in 2D (Table 3). Chymopapain treatment resulted in higher yield (total cells), improved cell viability, and reduced aggregate formation compared to papain treatment.

TABLE 3
Evaluation of late-stage CM cluster dissociation

| | Chymopapain | Papaya latex Papain |
|---|---|---|
| Total cells (cells/mL) | 724250 | 420250 |
| Cell viability (%) | 63.3 | 46.2 |
| Aggregates (%) | 0.75 | 10.5 |
| Cell diameter (μm) | 13.8 | 12.1 |

### REFERENCES

1) ChemoMetec, Application Note No. 2026. Rev. 1.4. Count & Viability—Via2-Cassette™
2) ChemoMetec, Application Note No. 2028. Rev. 1.3. Aggregated Cells—Via2-Cassette™

1-3. (canceled)

4. A method of cell detachment, comprising contacting a cell with a polypeptide having a sequence identity of at least 70% to SEQ ID NO:1, a mature polypeptide of SEQ ID NO:1, SEQ ID NO:2, or SEQ ID NO:3, wherein the polypeptide has chymopapain activity, wherein the cell is attached to a surface and/or to another cell.

5. The method according to claim 4, wherein the polypeptide comprises, consists essentially of, or consists of a mature polypeptide of SEQ ID NO:1.

6. The method according to claim 4, wherein the cell is a cardiomyocyte.

7. A composition suitable for cell detachment comprising a polypeptide having a sequence identity of at least 70% to SEQ ID NO:1, a mature polypeptide of SEQ ID NO:1, SEQ ID NO:2, or SEQ ID NO:3, wherein the polypeptide has chymopapain activity.

8. The composition according to claim 7, wherein the polypeptide comprises, consists essentially of, or consists of a mature polypeptide of SEQ ID NO:1.

9. A polynucleotide encoding a polypeptide having a sequence identity of at least 70% to SEQ ID NO:1, a mature polypeptide of SEQ ID NO:1, SEQ ID NO:2, or SEQ ID NO:3, wherein the polypeptide has chymopapain activity.

10. The polynucleotide according to claim 9, wherein the polypeptide comprises, consists essentially of, or consists of a mature polypeptide of SEQ ID NO:1.

11. A recombinant host cell transformed with a polynucleotide of claim 9.

12. The recombinant host cell according to claim 12, which is an Aspergillus cell.

13. A method of producing a polypeptide, comprising (a) cultivating a recombinant host cell according to claim 11 under conditions suitable for expression of the polypeptide; and (b) recovering the polypeptide.

14. The method according to claim 13, wherein the polypeptide comprises, consists essentially of, or consists of a mature polypeptide of SEQ ID NO:1.


SEQUENCE LISTING (6 sequences)

SEQ ID NO: 1 - protein, 352 aa, Carica papaya
MATMSSISKIIFLATCLIIHMGLSSADFYTVGYSQDDLTSIERLIQLFDSWMLKHNKIYESIDEKIYRFEIFRDNLMYIDETNKKNNSYWLGLNGFADLSNDEFKKKYVGFVAEDFTGLEHFDNEDFTYKHVTNYPQSIDWRAKGAVTPVKNQGACGSCWAFSTIATVEGINKIVTGNLLELSEQELVDCDKHSYGCKGGYQTTSLQYVANNGVHTSKVYPYQAKQYKCRATDKPGPKVKITGYKRVPSNCETSFLGALANQPLSVLVEAGGKPFQLYKSGVFDGPCGTKLDHAVTAVGYGTSDGKNYIIIKNSWGPNWGEKGYMRLKRQSGNSQGTCGVYKSSYYPFKGFA

SEQ ID NO: 2 - protein, 219 aa, Carica papaya
NYPQSIDWRAKGAVTPVKNQGACGSCWAFSTIATVEGINKIVTGNLLELSEQELVDCDKHSYGCKGGYQTTSLQYVANNGVHTSKVYPYQAKQYKCRATDKPGPKVKITGYKRVPSNCETSFLGALANQPLSVLVEAGGKPFQLYKSGVFDGPCGTKLDHAVTAVGYGTSDGKNYIIIKNSWGPNWGEKGYMRLKRQSGNSQGTCGVYKSSYYPFKGFA

SEQ ID NO: 3 - protein, 218 aa, Carica papaya
YPQSIDWRAKGAVTPVKNQGACGSCWAFSTIATVEGINKIVTGNLLELSEQELVDCDKHSYGCKGGYQTTSLQYVANNGVHTSKVYPYQAKQYKCRATDKPGPKVKITGYKRVPSNCETSFLGALANQPLSVLVEAGGKPFQLYKSGVFDGPCGTKLDHAVTAVGYGTSDGKNYIIIKNSWGPNWGEKGYMRLKRQSGNSQGTCGVYKSSYYPFKGFA

SEQ ID NO: 4 - DNA, 1038 bp, Synthetic construct
  source 1..1038: mol_type = other DNA
atgaaattgtcgtggttggtagctgctgcattgacggcagcatcagtcgtgtctgccgatttctacaccgtcggctacagccaggacgatcttacttcgatcgagagactcatccaacttttcgactcatggatgttgaagcacaataagatttacgaatcaattgacgagaagatttacagattcgagattttccgtgacaatcttatgtatatcgacgaaaccaacaagaagaataactcttactggctaggcctgaacggcttcgcggacctatcaaacgatgagttcaagaagaaatacgtgggcttcgtagccgaagactttacgggtctcgagcactttgataacgaagactttacgtacaaacatgtgaccaactacccccagtccatcgactggagggctaagggtgcggtcactcctgtcaagaaccaaggtgcctgcggcagttgctgggctttcagtacgatcgcgaccgttgagggaatcaacaaaatcgttacgggcaacctcctggagctttcggagcaggagctggtggactgcgacaagcatagctatggctgcaaagggggttatcagaccacttcactccagtacgttgccaacaatggagtccataccagcaaggtgtatccgtatcaagccaagcagtataagtgtcgggcaaccgataagcctggtcctaaggttaagatcacaggatacaagagagtccccagtaactgtgagacgtcgtttctcggagcacttgccaaccagccgttgtccgtcctggtcgaagccggaggcaagcctttccagctatacaagtcaggagtcttcgatggaccctgtggtacgaagctcgaccacgcggtcacagcagttggttacggcacctcggatggaaagaactatatcatcatcaagaattcgtggggtccaaactggggtgagaagggctatatgcgccttaaacgacaatctggtaactcccaagggacctgcggagtgtacaaatcatcgtactatcccttcaagggtttcgcttaa

SEQ ID NO: 5 - protein, 19 aa, Aspergillus oryzae
MKLSWLVAAALTAASVVSA

SEQ ID NO: 6 - protein, 4 aa, Synthetic construct
AAPF